(19) United States
(12) Patent Application Publication
(10) Pub. No.: US 2007/0055165 A1
(43) Pub. Date: Mar. 8, 2007

(54) Heart beat signal analysis

(76) Inventors: Jan Alexis Robrecht D'Aubioul, Mol (BE); Mario Jozef Joanna Peeters, Meerhout (BE); Karel Jacobus Mathilda Van Ammel, Beerse (BE)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 10/573,224

(22) PCT Filed: Sep. 22, 2004
(86) PCT No.: PCT/EP04/10657

§ 371 Date: Mar. 24, 2006

(30) Foreign Application Priority Data
Sep. 26, 2003 (EP) 03078036.5

(51) Int. Cl. A61B 5/04

(52) U.S. Cl. 600/509

(57) ABSTRACT

There are disclosed methods and apparatus for analysing heart beat signals which comprise a series of similar heart beat complexes, such as ECG signals. A subset of complexes satisfying a mutual similarity criterion is selected from a larger set. A representative complex is calculated from the members of the subset. One or more heart beat signal parameters are then established from the representative complex. Computer apparatus making use of this scheme, including a graphical user interface for validating the representative complexes, is also disclosed.

The present invention relates to methods and apparatus for analysing quasi-repetitive signals comprising a series of similar complexes, and in particular, but not exclusively, to such methods and apparatus suitable for analysing heart beat signals such as electrocardiograph signals.

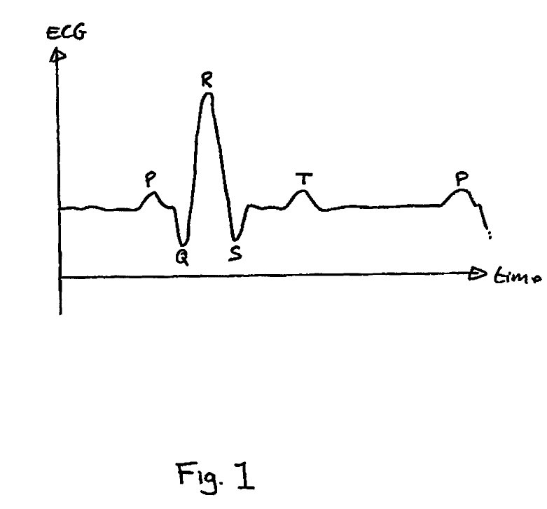

An electrocardiogram (ECG) is a graph of the voltage variations plotted against time. The variations results from the depolarisation and depolarisation of the cardiac muscle, which produces electrical fields that reach the surface of the body where electrodes are located Placement of multiple electrodes at selected points on the body enables a variety of different signals to be picked up simultaneously (multiple lead electrocardiography), from which a reasonably complete picture of the electrical activity of the heart, and any related clinical abnormalities can be deduced. A heart beat complex from a typical ECG signal is shown in simplified form in FIG. 1. The signal comprises many such complexes in series, each corresponding to one heart beat. An ECG signal may be taken from each lead. Typically, twelve leads are used.

The principal features of an ECG complex have widely accepted designations. An initial P-wave is followed by a larger QRS-wave. A T-wave follows the QRS-wave. Various minor variations in the shape of a complex, as well as missed and extra beats are expected in a healthy subject. Heart abnormalities, diseases, and drugs also affect the shape and timing of the features of a complex. Trials of a drug generally include experiments to determine any such changes, from which the effect of a drug on the electrophysiological activity of the heart can be deduced.

Pre-clinical drug trials generally include administering a drug to animals using various dosage schemes. During an experiment, which typically lasts several hours, one or several doses of a drug may be administered and ECG recordings are made. It is time consuming to manually analyse the large amount of ECG data resulting from such experiments, and from other situations in which long series of heart beats are recorded.

Automatic analysis of ECG complexes to identify various parameters is well known. Analysis of the mutual timings of various features of signals from one or more ECG leads, for example, is routinely built into ECG machines for clinical use. Such shape analysis is usually carried out on single selected heart beat complexes. Automatic shape analysis of the average of a series of complexes is also known, for example from U.S. Pat. No. 5,139,027, which describes an apparatus which also automatically looks for features in an ECG signal such as narrow spikes, electrical noise and other artifacts and discards them before forming an average of a plurality of complexes. The averaging of a plurality of complexes before performing shape analysis is advantageous in that it reduces the computational cost of the expensive shape analysis process and improves the signal to noise ratio of the analysed signal.

However, the known schemes for automatic analysis of ECG signals are not ideal in many situations, such as pre-clinical drug trials, because they do not adequately allow for natural variations in shape and timing between the members of a group of heart beat complexes to be averaged.

The invention seeks to address the above and other problems of the related prior art.

Accordingly, the invention provides a method of analysing a heart beat signal which comprises a series of heart beat complexes, the method comprising the steps of:

selecting a set of said complexes;

identifying from the set a subset of complexes each of which satisfy a similarity criterion with respect to members of the set;

calculating a representative complex from the members of the subset; and

establishing one or more heart beat signal parameters from said representative complex.

The heart beat signal is preferably an ECG signal, but the method may also be applied to other types of heart beat signal, such as a signal from a pulse oximeter, a thoracic motion sensor or an ultrasound device. Each heart beat complex preferably corresponds to a single heart beat, or portion thereof. Each selected set of complexes preferably includes only genuine complexes, excluding artifacts of various sorts familiar to the skilled person, such as spikes and electrical noise. The complexes in any one set are preferably consecutive within said signal. Typically, the set will comprise between about two and twenty complexes, and preferably between four and ten complexes.

The heart beat signal parameters may be calculated from the representative complex using a variety of techniques known in the art. For an ECG signal, parameters such as the PQ, QRS and QT intervals, and parameters derived from intervals between features on different leads may be calculated.

Preferably, the step of identifying comprises identifying from the set a subset of complexes each of which satisfy a similarity criterion with respect to an average of the members of the set, where the average could be a mean, an unnormalised sum, or a variety of other types of average. Preferably, said step of calculating comprises calculating the average complex of the members of the subset, where again the average could be one of several types of average.

Preferably, the method includes a step of analysing said heart beat signal to identify and select genuine, or valid heart beat complexes to form the above mentioned series, before any of steps mentioned above are carried out. The method may also include steps of aligning the complexes of the series, the set and/or the subset prior to operations such as correlation and averaging.

Preferably, the similarity criterion comprises a threshold calculated using the complexes of said set and an average of these complexes. For example, the criterion could be based on coefficients of correlation between each member of the set and the average of the set, the criterion comprising a threshold correlation coefficient. Such a threshold coefficient may itself be based on the distribution of the calculated correlation coefficients for the set. However, the similarity criteria preferably also comprises a predefined threshold value, for example a fixed correlation coefficient of 0.98, above which the criterion is always satisfied. This ensures that no complexes fail the similarity test if they are all very similar.

Said step of identifying may further comprise aligning the complexes of said set, forming a set-average complex from said aligned complexes, comparing each complex of said set to said set-average complex,

determining if each result of comparing satisfies said similarity criterion, and forming said subset from only those complexes of said set for which the similarity criterion is satisfied. Preferably, the step of comparing comprises correlating each complex of said set with said set-average complex.

If said signal is an electrocardiograph signal, said step of aligning the complexes of said set preferably comprises the step of aligning said complexes on an R-top feature of each complex, although other alignment schemes could also be used.

The method steps described above may conveniently be implemented using computer software for execution on a suitable computer system. Such software may conveniently be written on one or more computer readable media such as CDROMs. The invention also provides a computer system arranged to carry out the methods. Such a computer system may provide, for example, an extract set element adapted to select a set of complexes from the series of complexes to be analysed, a select subset element adapted to identify from the set a subset of complexes each of which satisfy a similarity criterion with respect to members of the set, and a combiner element adapted to calculate a representative complex from the members of the subset.

The invention also provides apparatus for analysing a heart beat signal comprising a plurality of heart beat complexes, the apparatus comprising: an analysis engine adapted to automatically calculate a representative heart beat complex for each of a plurality of preselected intervals of said signal; and

an editor adapted to enable a user to edit calculation and/or heart beat parameters relating to each automatically calculated representative heart beat complex. Typically, each interval contains a set of heart beat complexes. The analysis engine is preferably adapted to automatically decide which complexes in the set to include in each representative heart beat complex, and the editor is preferably adapted to enable the user to subsequently change which complexes are included.

The editor may enable the user to control calculation parameters which determine whether a particular complex should be included, not included, or included subject to an automatic or predefined similarity criterion, in the corresponding representative heart beat complex. This representative complex may be an average of the included complexes.

The editor may further provide the user with a graphical display of at least some of the heart beat complexes within a current one of the intervals of said signal, and with a graphical display of the current representative heart beat complex for the current interval. In this way, the user may be provided with facilities to validate the representative heart beat complex and correct it if necessary.

For example, the editor may be further adapted to display, on the display of the current representative heart beat complex, one or more feature markers indicating the locations of one or more automatically determined features of said representative heart beat complex, such as P, Q, R, S and T features of an ECG complex. The editor is preferably further adapted to enable the user to move said markers, for example by means of a drag and drop action using a pointer device.

Preferably, the analysis engine is adapted to calculate one or more heart beat parameters, especially shape parameters, of each representative heart beat complex, and said editor is adapted to display said heart beat parameters and to update said display according to changes made by said user. Such heart beat parameters could include, for ECG analysis, the duration of the QT, PQ and QRS as well as of other intervals between features for a single lead or between leads.

The apparatus is preferably provided by means of a computer program and appropriate data elements, which may be provided on one or more removable computer readable media, executing on a computer. One preferred embodiment is implemented using Microsoft Excel® spreadsheet templates, macros and other appropriate data structures and files.

The method and apparatus may be used in a variety of circumstances and applications. In the embodiments set out below, pre-clinical drug trials are particularly mentioned, but other applications include any analysis of heart beat data including clinical drug trials, medical check ups, patient monitoring in intensive care, during and after surgical procedures, in athletic activity monitors and so on.

The methods and apparatus may also be applied to repetitive or quasi-repetitive signals other than heart beat signals including to signals of medical origin and use and to non-medical signals.

Embodiments of the invention will now be described, by way of example only, with reference to the accompanying drawings, of which:

FIG. 1 shows features of a typical ECG signal;

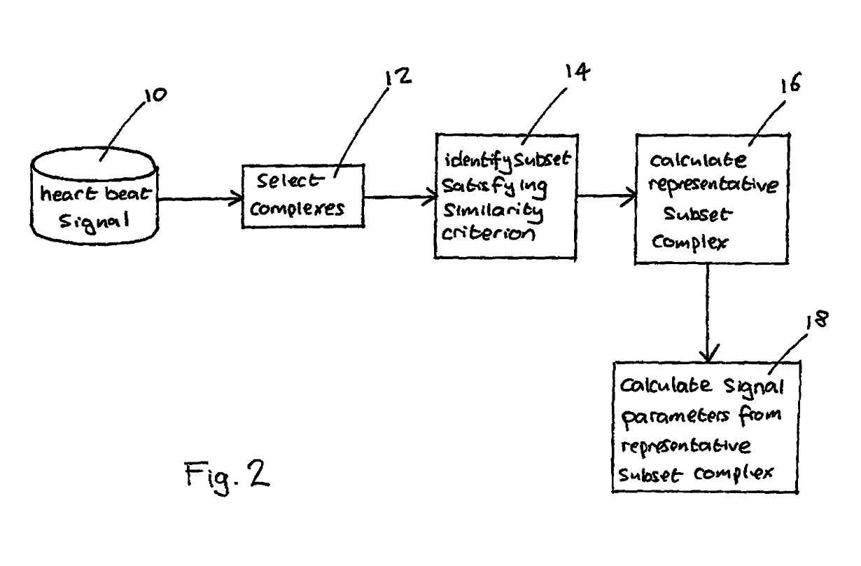

FIG. 2 sets out a scheme for analysing a heart beat signal;

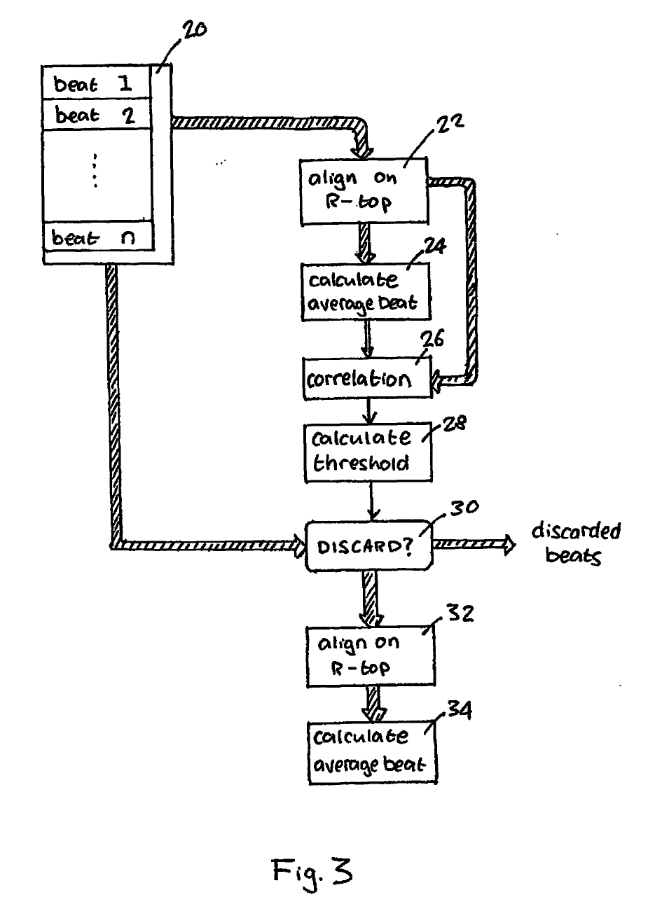

FIG. 3 illustrates how steps 14 and 16 of FIG. 2 may be put into effect, in more detail;

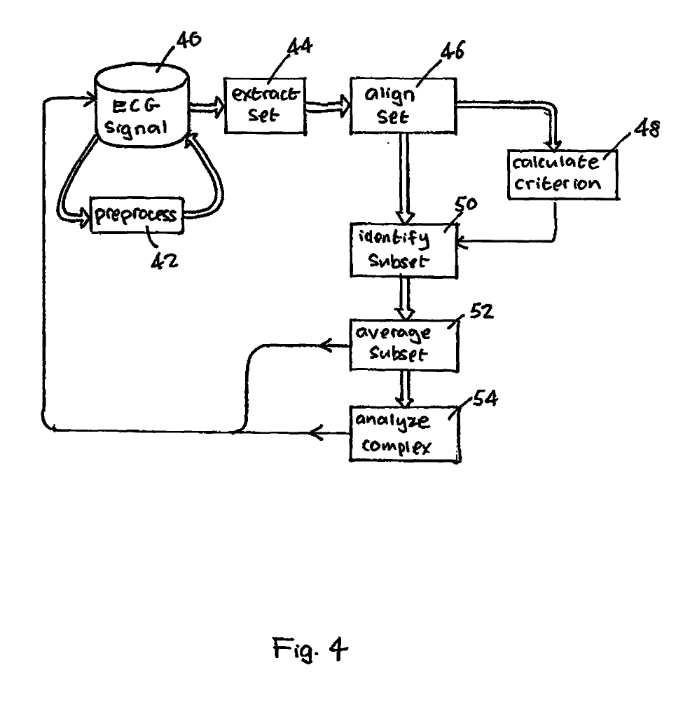

FIG. 4 illustrates apparatus and/or computer program elements for putting the method steps of FIGS. 2 and 3 into effect;

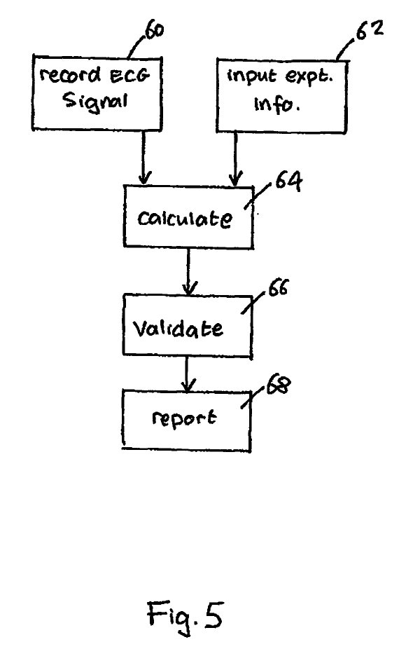

FIG. 5 sets out a scheme for analysis and validation of a heart beat signal; and

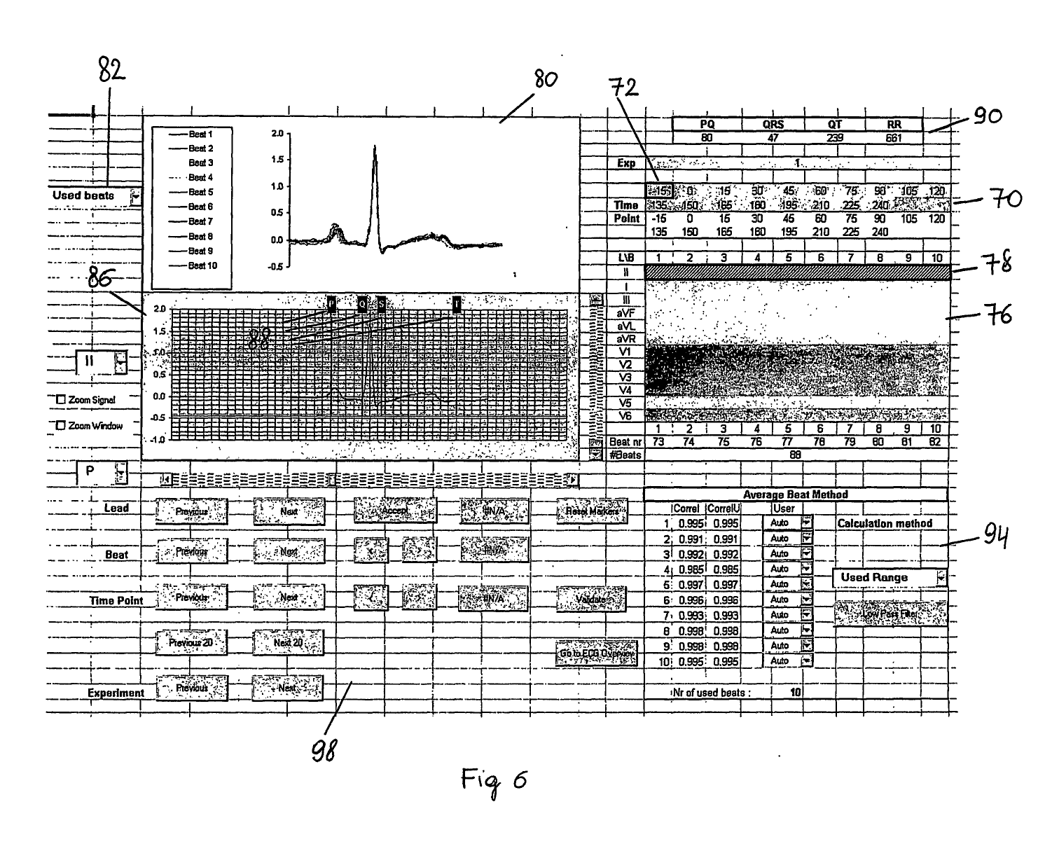

FIG. 6 shows a graphical user interface for assisting in the validation step of FIG. 5.

FIG. 2 illustrates, schematically, a method of analysing a heart beat signal 10, such as an ECG signal, embodying the invention. The method comprises steps of selecting a set of heart beat complexes from within the signal (step 12), identifying a subset of those complexes which all match a quality, similarity or mutual similarity criterion (step 14) and calculating an average or representative complex of the identified subset (step 16). The average complex may be stored and used to calculate one or more heart beat parameters (step 18). If the signal is an ECG signal, then these heart beat parameters may be, for example, a QRS interval, a PQ interval, a QT interval and so on.

Steps 14 and 16 of FIG. 2 may be implemented as illustrated in FIG. 3. The members of the selected set of heart beat complexes 20 are first aligned in step 22. The alignment may conveniently be carried out using a single dominant feature such as the R-peak of an ECG complex, or may involve optimization of alignment of more then one feature or region of each complex. After alignment, the complexes are then summed or averaged in step 24. A correlation coefficient between each aligned complex and the averaged complex is then calculated in step 26. The set of correlation coefficients so obtained is used to calculate a threshold parameter in step 28.

The threshold parameter calculated in step 28 is used in step 30 to discard one or more of the original complexes 20 according to a similarity criterion using the parameter. The remaining subset of complexes may again be aligned, for example with reference to an R-top, in step 32, and the subset is then used to calculate an average complex, representative of the original complexes 20, in step 34.

A suitable threshold parameter calculated in step 28 is the value above which 95% of the correlation coefficients would be expected to lie, based on the average and standard deviation of their actual values. The similarity criterion used in step 30 could then be such as to discard complexes having a correlation coefficient calculated in step 26 falling below this threshold parameter, subject to a maximum threshold parameter value of 0.98.

The method illustrated in FIGS. 2 and 3 may be put into effect on any suitable computer equipment, in particular using suitable software elements which may be provided on one or more computer readable media. Suitable elements of such software, or computer apparatus suitably programmed, are illustrated in FIG. 4. The arrangement may be controlled and the results of the implemented process displayed using the apparatus and user interface further described below in relation to FIG. 6.

FIG. 4 illustrates a memory device 40, such as a disk drive or RAM, storing an ECG or other heart beat signal. The signal may be preprocessed in various ways by preprocess element 42. The preprocess element 42 may carry out cleaning and filtering processes on the signal, and in particular may identify and label valid heart beat complexes. Noise artifacts and badly distorted complexes are rejected, for example by comparison against templates representing acceptable complex shapes, by checking the timing of each complex within the signal, or by other techniques known in the prior art.

An extract element 44 selects one or more sets of complexes for further analysis, each set preferably containing a fixed number of consecutive complexes. For the purposes of pre-clinical drug trials each set could be a series of ten complexes selected at fifteen minute intervals throughout an experiment, with extra sets being selected in each period immediately following a drug administration.

An align element 46 aligns the complexes of each set. A calculate criterion element 48 calculates the parameter or parameters, such as a threshold, for deciding which complexes of the set to use further, and which to reject, for example as described above in connection with steps 24, 26 and 28 of FIG. 3. An identify subset element 50 uses the parameter or parameters from the calculate criterion element 48 to select a subset of the complexes, as described above in connection with step 30 of FIG. 3. Finally, the selected subset is used by averaging element 52 to calculate a representative complex, and the analyse complex element 54 automatically analyses the average complex to obtain desired heart beat parameters. At each stage of the process, and especially at the final analysis stage, results may be written back to the memory 40 in a manner so to be suitably associated with the original signal data.

Generally, and particularly for pre-clinical drug trials, the heart beat signal analysis scheme described above is incorporated into a process such as that set out in FIG. 5. An ECG signal is recorded at step 60. Corresponding experiment information is input at step 62, and used to perform an automatic analysis of the ECG signal at step 64. The results of the automatic analysis are checked, and if necessary adjusted at validation step 66, and finally an experiment report is produced at step 68. The experiment information may include conditions of the experiment, such as time, date, duration, location, subject animal, drug, dosage and so on, as well as parameters expressly constraining the subsequent calculation step 64 such as the time points at which detailed analysis is required, how that analysis is to be carried out, and what result heart beat parameters are to be obtained.

In one embodiment of the invention, steps 62 to 68 of FIG. 5 are implemented using Microsoft Excel® spreadsheet templates and macros, with ECG signal data being imported in a compatible file format. FIG. 6 illustrates a graphical user interface provided as part of this embodiment to implement the validation and adjustment step 66 of FIG. 5 following the calculation step 64. This interface is displayed by a Microsoft Excel® tool executing on a conventional personal computer, providing conventional pointing device, keyboard and visual display peripherals.

The duration of the experiment being validated in the interface of FIG. 6 is shown in the time point area 70. The time points (in minutes) at which detailed analysis of the ECG signal is required are shown. The first time point is minus fifteen minutes, because the zero minute point is the time of a first drug administration. A current time point is indicated by a box 72 surrounding the “−15” value. Completed validation at a time point is indicated by colour coding in the time point area 70.

The ECG parameters being validated are based upon up to twelve time-parallel signals from up to twelve ECG leads, listed in the left hand column of a validation table 76. A current ECG lead is indicated by a box 78 surrounding the lead II row. Colour coding in the validation table 76 illustrates which beats of which lead have been validated, and which leads do not require validation. A protocol editor may be available. In said protocol editor the user can predefine time points, leads, parameters and so on that are to be used or calculated in the ECG editor. The horizontal axis of the lead table is representative of the ten beats (or other number as appropriate) to be averaged at the current time point.

The ECG signal of the beats to be used at the current time point for the current lead are plotted in a beat plot area 80, as multiple curves overlaid according to an automatic alignment process. The ten beats shown in the beat plot area 80 of FIG. 6 have been aligned on their R-top features. Which beats are plotted can be controlled using a beat plot selector 82, which currently indicates that all beats of the set except beats which have been rejected on the basis of a similarity test (ie used beats) are shown. Other options provided by selector 82 are all beats of the set, and single selected beats.

Below the beat plot area is an average beat plot area 86 which displays, using a horizontal axis aligned with that of the beat plot area 80, an average beat calculated according to the method illustrated in FIGS. 2 to 4. Also shown are markers 88 (P, Q, S and T) showing the positions of corresponding features of the average beat which have been automatically calculated, for example using beat shape analysis techniques known in the prior art. The markers 88, which could mark various different features, can be moved by the user. Heart beat parameters derived from the current marker positions are given in a heart beat parameter area 90.

Although the average beat and consequent heart beat parameters are automatically calculated for all time points according to a schedule included with the information provided in step 62 of FIG. 5, the user of the interface of FIG. 6 can subsequently validate and intervene in the beat averaging process by means of the controls provided in the average beat control area 94. Each row of this area corresponds to one of the beats at the current time point. The “Correl” column shows the correlation coefficient of each beat, once aligned, with the average of all beats of the set, and the “Correl U” column shows the correlation of each beat, once aligned, with the average of all beats currently used to form the average beat shown in the average beat plot area 86.

The “User” column of the average beat control area 94 enables the user to allow the tool to decide whether to include a beat in the average according to the predetermined scheme, or to override the scheme and choose to include or exclude the beat from the average. This facility allows a user to reject a distorted or otherwise incorrect or undesirable beat, and to include an otherwise rejected beat if necessary.

Navigation area 98 provides facilities for moving the current validation point between leads, beats, time points and experiments, for accepting and validating data and derived parameters and so on.

Alternatives and variations to the embodiments described above will be apparent to the person skilled in the art, while still falling within the scope of the appended claims and their equivalents. For example, a variety of different programming languages could be used to implement the invention, on a variety of different hardware platforms including dedicated ECG machines, portable heart beat monitors, and conventional computer work stations. A validation interface could include various combinations of graphical elements selected from those illustrated in FIG. 6, as well as other elements. The validation interface may also be provided as a separate entity to any automatic processing means, the results from which it is to be used to validate.

Various criteria may be used to select which heart beat complexes to include when calculating a representative complex. In some applications only complexes deviating to a large extent from the average of a set will be rejected, while in other applications only near identical complexes will be included.

1. A method of analysing a heart beat signal which comprises a series of heart beat complexes, the method comprising the steps of:
selecting a set of said complexes;
identifying from the set a subset of complexes each of which satisfy a similarity criterion with respect to members of the set;
calculating a representative complex from the members of the subset; and
establishing one or more heart beat signal parameters from said representative complex.

2. The method of claim 1 wherein said step of identifying comprises identifying from the set a subset of complexes each of which satisfy a similarity criterion with respect to an average of the members of the set.

3. The method of claim 2 wherein said similarity criterion comprises a threshold calculated using the complexes of said set and an average of the members of the set.

4. The method of claim 3 wherein said similarity criterion further comprises a predefined threshold value.

5. The method of claim 1 wherein said step of calculating a representative complex is a step of calculating an average complex of the members of the subset.

6. The method of claim 1 further comprising a step of analysing said heart beat signal to identify and select genuine heart beat complexes to form said series.

7. The method of claim 1 wherein said step of identifying comprises:
aligning the complexes of said set;
forming a set-average complex from said aligned complexes;
comparing each complex of said set to said set-average complex;
determining if each result of comparing satisfies said similarity criterion; and
forming said subset from only those complexes of said set for which the similarity criterion is satisfied.

8. The method of claim 7 wherein said step of comparing comprises correlating each complex of said set with said set-average complex, and comparing each resulting correlation coefficient with a threshold value.

9. The method of claim 7 wherein said signal is an electrocardiogram signal and wherein said step of aligning the complexes of said set comprises the step of aligning said complexes on an R-top feature of each complex.

10. The method of claim 1 wherein said heart beat signal is an electrocardiogram signal.

11. Computer apparatus adapted to carry out the method steps of claim 1.

12. A computer readable medium carrying a computer program product comprising elements adapted to carry out the method steps of claim 1.

13. Apparatus for analysing a heart beat signal which comprises a series of heart beat complexes, comprising:
an extract set element (44) adapted to select a set of complexes from said series;
a select subset element (46, 48, 50) adapted to identify from the set a subset of complexes each of which satisfy a similarity criterion with respect of members of the set; and
a combiner element (52) adapted to calculate a representative complex from the members of the subset.

14. The apparatus of claim 13 further comprising an analysis element (54) adapted to establish one or more heart beat signal parameters from said representative complex.

15. The apparatus of claim 13 wherein the select subset element comprises:
an align set element (46) adapted to align the complexes of said set; and
a calculate criterion element (48) adapted to establish said similarity criterion from the aligned complexes.

16. The apparatus of claim 15 wherein the calculate criterion element (48) establishes said similarity criterion as a threshold calculated using the complexes of said set and an average of the members of the set.

17. The apparatus of claim 13 further comprising a preprocess element (42) adapted to identify and select genuine heart beat complexes to form said series.

18. Apparatus for analysing a heart beat signal comprising a plurality of heart beat complexes, the apparatus comprising:
an analysis engine adapted to automatically calculate a representative heart beat complex for each of a plurality of preselected intervals of said signal; and
an editor adapted to enable a user to edit parameters relating to each automatically calculated representative heart beat complex.

19. The apparatus of claim 18 wherein each interval contains a set of heart beat complexes, the analysis engine is adapted to automatically decide which complexes in the set to include in each representative heart beat complex, and the editor is adapted to enable the user to subsequently change which complexes are included.

20. The apparatus of claim 19 wherein the editor is adapted to enable the user to select whether a particular complex should be included, not included, or included subject to a similarity criterion, in the corresponding representative heart beat complex.

21. The apparatus of claim 18 wherein the representative complex is an average of the included complexes.

22. The apparatus claim 18 wherein the editor is further adapted to provide the user with a graphical display of at least some of the heart beat complexes within a current one of the intervals of said signal and with a graphical display of the current representative heart beat complex for the interval.

23. The apparatus of claim 22 wherein the editor is further adapted to display, on the display of the current representative heart beat complex, one or more feature markers indicating the automatically determined locations of one or more features of said representative heart beat complex.

24. The apparatus of claim 23 wherein the editor is further adapted to enable the user to move said markers.

25. The apparatus of claim 18 wherein the analysis engine is further adapted to calculate one or more heart beat parameters of each representative heart beat complex, and said editor is adapted to display said heart beat parameters and to update said display according to changes made by said user.

26. A computer readable medium comprising a computer program product arranged to provide the apparatus of claim 18 when executed on a suitable computer system.
